(19) United States
(12) Patent Application Publication
(10) Pub. No.: US 2012/0029366 A1
(43) Pub. Date: Feb. 2, 2012

(54) BLOOD PRESSURE DETECTION APPARATUS AND BLOOD PRESSURE DETECTION METHOD

(75) Inventors: Toshihiko YOKOYAMA, Matsumoto (JP); Osamu TOCHIKUBO, Yokohama (JP)

(73) Assignees: PUBLIC UNIVERSITY CORPORATION YOKOHAMA CITY UNIVERSITY, Yokohama (JP); SEIKO EPSON CORPORATION, Tokyo (JP)

(21) Appl. No.: 13/191,584

(22) Filed: Jul. 27, 2011

(30) Foreign Application Priority Data
Aug. 2, 2010 (JP) 2010-173377

(51) Int. Cl. A61B 5/021

(52) U.S. Cl. 600/490

(57) ABSTRACT

A blood pressure detection apparatus includes a pressure sensor, a press mechanism that applies pressure to a blood vessel by pressing a living body, and can gradually decrease the pressure, and a blood pressure calculation section that determines the pressure when a given waveform pattern appears in a waveform of pulse waves obtained by the pressure sensor to be a maximal blood pressure, determines the pressure when the waveform has a maximum amplitude to be a mean blood pressure, and calculates a minimal blood pressure using the maximal blood pressure and the mean blood pressure.

Japanese Patent Application No. 2010-173377 filed on Aug. 2, 2010, is hereby incorporated by reference in its entirety.

### BACKGROUND

The blood pressure may be non-invasively measured by an auscultatory method or an oscillometric method.

When externally applying pressure equal to or higher than the maximal blood pressure to an artery, and gradually decreasing the applied pressure, the artery produces vibrations in the audible region (Korotkoff's sound) within a specific pressure range. The auscultatory method determines the applied pressure when the Korotkoff's sound is produced to be the maximal blood pressure, and determines the applied pressure when the Korotkoff's sound stops to be the minimal blood pressure.

The oscillometric method utilizes a phenomenon in which the dynamic properties of an arterial wall nonlinearly change relative to externally applied pressure. The diameter and the volume of a blood vessel change with each heartbeat. The change in volume differs depending on the pressure inside the blood vessel (blood pressure) and the externally applied pressure, but shows significant nonlinearity with respect to the internal-external pressure difference (tube law). When applying pressure equal to or higher than the maximal blood pressure to a blood vessel, the blood vessel is occluded (i.e., a change in volume does not occur). When gradually decreasing the applied pressure, a change in volume of the blood vessel occurs when the applied pressure becomes lower than the maximal blood pressure, becomes a maximum when the applied pressure is almost equal to the mean blood pressure, and stops when the applied pressure is almost equal to the minimal blood pressure.

The applied pressure and the change in volume of the blood vessel are recorded at the same time during the above process to determine the maximal blood pressure, the mean blood pressure, and the minimal blood pressure.

For example, JP-A-2006-280485 discloses a method that easily obtains a pulse waveform that allows direct detection of the pulse waves from a living body using a pulse wave detection means provided with a blood pressure detection strain sensor. Since the pulse waves have wavelength characteristics with a notch, the pulse waves can be clearly distinguished from noise using a band-pass filter or the like, and the maximal blood pressure and the minimum blood pressure can be accurately detected by utilizing the pulse waves.

### SUMMARY

According to one aspect of the invention, there is provided a blood pressure detection apparatus comprising:

a press mechanism that applies pressure to a blood vessel by pressing a living body, and can gradually decrease the pressure;

a pressure sensor that detects a change in pressure of the blood vessel caused by a change in the pressure applied by the press mechanism; and

a blood pressure calculation section that determines the pressure applied by the press mechanism when a given waveform pattern appears in a waveform that indicates a change in pressure of the blood vessel to be a maximal blood pressure, determines the pressure applied by the press mechanism when the waveform has a maximum amplitude to be a mean blood pressure, and calculates a minimal blood pressure using the maximal blood pressure and the mean blood pressure.

According to another aspect of the invention, there is provided a blood pressure detection method comprising:

applying pressure to a blood vessel by pressing a living body;

gradually decreasing the pressure applied to the blood vessel;

detecting a change in pressure of the blood vessel caused by a change in the pressure applied to the blood vessel; and

determining the pressure applied to the blood vessel when a given waveform pattern appears in a waveform that indicates a change in pressure of the blood vessel to be a maximal blood pressure, determining the pressure applied to the blood vessel when the waveform has a maximum amplitude to be a mean blood pressure, and calculating a minimal blood pressure using the maximal blood pressure and the mean blood pressure.

### BRIEF DESCRIPTION OF THE DRAWINGS

FIG. 1 is a view illustrating a state in which a blood pressure detection apparatus is attached to a wrist.

FIG. 2 is a view illustrating a state in which a blood pressure detection apparatus is attached to a wrist.

FIG. 3 is a view illustrating the details of a press mechanism.

FIG. 4 is a view illustrating the configuration of a pressure sensor.

FIG. 5 is a view illustrating the details of a control-display section.

FIG. 6A is a view illustrating an oscillometric waveform, FIG. 6B is a view illustrating a differential waveform, and FIG. 6C is a view illustrating a pressure signal waveform.

FIG. 7 is a view illustrating an oscillometric waveform and a pressure waveform thereof.

FIGS. 8A and 8B are views illustrating an oscillometric waveform and a pressure waveform thereof.

FIG. 9 is a view illustrating an oscillometric waveform and a differential waveform thereof.

FIG. 10 is a view illustrating a systolic waveform pattern included in an oscillometric waveform.

FIGS. 11A to 11F are views illustrating a systolic waveform pattern included in an oscillometric waveform.

FIG. 12 is a flowchart illustrating the entire operation (process) according to one embodiment of the invention.

FIG. 13 is a view illustrating a systolic waveform pattern included in an oscillometric waveform according to a modification.

### DESCRIPTION OF EXEMPLARY EMBODIMENTS

In JP-A-2006-280485, a change in volume of the blood vessel with each heartbeat and the applied pressure are recorded during the entire process when applying pressure and decreasing the applied pressure, and features corresponding to the systolic phase, the mean value, and the diastolic phase are extracted from the change in volume to determine the systolic blood pressure, the mean blood pressure, and the diastolic blood pressure. Specifically, when determining the blood pressure based on the oscillometric method as disclosed in JP-A-2006-280485, it is necessary to record a change in volume and the applied pressure during the entire measurement process (i.e., the blood vessel starts to vibrate, a change in volume becomes a maximum, and a change in volume stops) to acquire the entire change in volume of the blood vessel. Since a change in volume cannot be accurately determined if the applied pressure is reduced too quickly, it is necessary to reduce the applied pressure over a period equal to or longer than about 20 heartbeats in order to accurately determine the blood pressure. If the heartbeat cycle is 1 second, it takes about 20 seconds. Therefore, it takes about 30 seconds (including the pressure-applying process) in order to accurately measure the blood pressure.

The blood pressure is defined as the value in the origin of the aorta. When the measurement site is at a height of 10 cm from the heart, an error occurs by about 7.5 mmHg. Therefore, it is necessary to maintain the measurement site at the height of the heart during the measurement. Accordingly, it is necessary to maintain the measurement site at the height of the heart for several tens of seconds during normal blood pressure measurement.

A humerus-type sphygmomanometer or a wrist-type sphygmomanometer currently on the market are normally used several times a day (e.g., morning, daytime, and night).

It is obvious that the number of patients who suffer from cardiac diseases or circulatory diseases (e.g., cerebrovascular disorder) will increase along with aging of the population in the future. Therefore, it will be necessary to more carefully manage the blood pressure for prevention or prognosis of these diseases. Accordingly, a wearable sphygmomanometer that allows arbitrary blood pressure measurement is desired. However, the current technology requires the user to hold the body for 30 seconds or more during blood pressure measurement, for example. This is very inconvenient to the user.

Several embodiments of the invention may provide a blood pressure detection apparatus and a blood pressure detection method that can reduce the time required for blood pressure measurement.

According to one embodiment of the invention, there is provided a blood pressure detection apparatus comprising:

a press mechanism that applies pressure to a blood vessel by pressing a living body, and can gradually decrease the pressure;

a pressure sensor that detects a change in pressure of the blood vessel caused by a change in the pressure applied by the press mechanism; and

a blood pressure calculation section that determines the pressure applied by the press mechanism when a given waveform pattern appears in a waveform that indicates a change in pressure of the blood vessel to be a maximal blood pressure, determines the pressure applied by the press mechanism when the waveform has a maximum amplitude to be a mean blood pressure, and calculates a minimal blood pressure using the maximal blood pressure and the mean blood pressure.

The inventors of the invention found by experiments that the applied pressure (pressure applied by the press mechanism) when a given waveform pattern appears in the waveform that indicates a change in pressure of the blood vessel corresponds to the maximal blood pressure. Therefore, the pressure applied by the press mechanism when a given waveform pattern is observed can be determined to be the systolic blood pressure (maximal blood pressure) by merely determining the presence or absence of the given waveform pattern instead of determining the change in volume of the blood vessel during the entire pressure application process. This makes it possible to reduce the measurement time as compared with a related-art blood pressure determination method.

In the blood pressure detection apparatus,

the given waveform pattern may be a waveform that indicates pulse waves that include a first maximum value, and a second maximum value that is obtained when the pressure applied by the press mechanism is lower than that when the first maximum value is obtained, the second maximum value being larger than the first maximum value.

The inventors found by experiments that the applied pressure when a waveform that indicates pulse waves that include a first maximum value, and a second maximum value when the pressure applied by the press mechanism is lower than that when the first maximum value is obtained, the second maximum value being larger than the first maximum value, is measured as the given waveform pattern corresponds to the maximal blood pressure. This makes it possible to easily detect the presence or absence of the given waveform pattern, and reduce the measurement time as compared with a related-art blood pressure determination method.

In the blood pressure detection apparatus,

the press mechanism may gradually open an artery from an occlusion state.

The inventors found by experiments that the given waveform pattern occurs when an artery is gradually opened from an occlusion state. Specifically, the given waveform pattern occurs by causing the press mechanism to apply pressure that occludes the artery, and then gradually decrease to the pressure so that the artery is opened. This makes it possible to easily provide a blood pressure detection apparatus that can reduce the measurement time as compared with a related-art blood pressure determination method.

In the blood pressure detection apparatus,

the press mechanism may gradually occlude an artery from an open state.

When using a related-art blood pressure determination method, since it is necessary to determine (observe) the change in volume of the blood vessel during the entire pressure application process, it is necessary to cause the press mechanism to gradually occlude the artery, and then gradually open the artery. Therefore, it takes time to determine the blood pressure. The inventor found by experiments that the given waveform pattern occurs when an artery is gradually occluded from an open state. Therefore, the maximal blood pressure can be determined based on the given waveform pattern that occurs when causing the press mechanism to gradually occlude the artery. This makes it possible to further reduce the measurement time as compared with a related-art blood pressure determination method.

In the blood pressure detection apparatus,

the artery may be a radial artery.

The radial artery is present at a shallow position from the body surface. Since the radius is present right under the radial artery, the pressure can be applied to the radial artery by the press mechanism without being dispersed. Therefore, the blood pressure can be more reliably detected by occluding and opening the radial artery using the press mechanism.

According to another embodiment of the invention, there is provided a blood pressure detection method comprising:

applying pressure to a blood vessel by pressing a living body;

gradually decreasing the pressure applied to the blood vessel;

detecting a change in pressure of the blood vessel caused by a change in the pressure applied to the blood vessel; and

determining the pressure applied to the blood vessel when a given waveform pattern appears in a waveform that indicates a change in pressure of the blood vessel to be a maximal blood pressure, determining the pressure applied to the blood vessel when the waveform has a maximum amplitude to be a mean blood pressure, and calculating a minimal blood pressure using the maximal blood pressure and the mean blood pressure.

The inventor found by experiments that the applied pressure (pressure applied to the blood vessel) when a given waveform pattern appears in the waveform that indicates a change in pressure of the blood vessel corresponds to the maximal blood pressure. Therefore, the applied pressure when a given waveform pattern is observed can be determined to be the maximal blood pressure by merely determining the presence or absence of the given waveform pattern instead of determining the change in volume of the blood vessel during the entire pressure application process. This makes it possible to reduce the measurement time as compared with a related-art blood pressure determination method.

Exemplary embodiments of the invention are described in detail below with reference to the drawings.

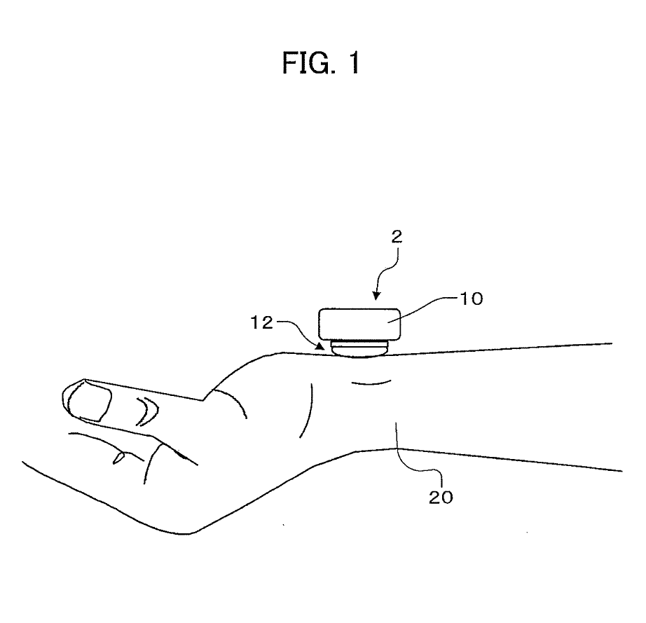

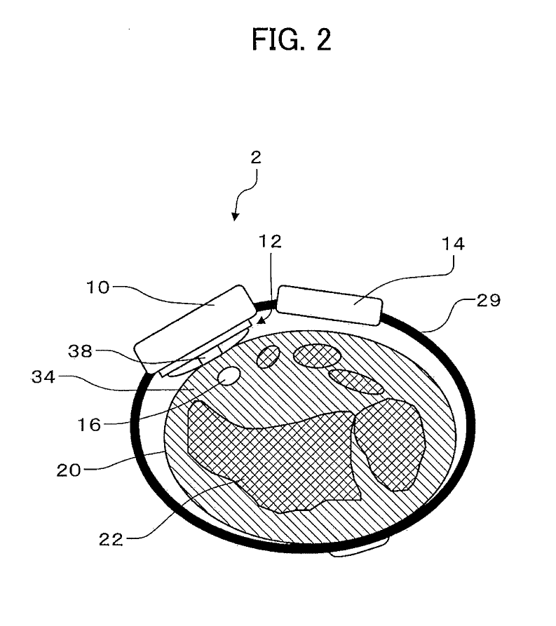

FIGS. 1 and 2 are views illustrating a state in which a blood pressure detection apparatus according to one embodiment of the invention is attached to a wrist. FIG. 1 is an external side view, and FIG. 2 is a cross-sectional view.

A blood pressure detection apparatus 2 according to one embodiment of the invention includes a press mechanism 10, a pressure sensor 12, and a control-display section 14.

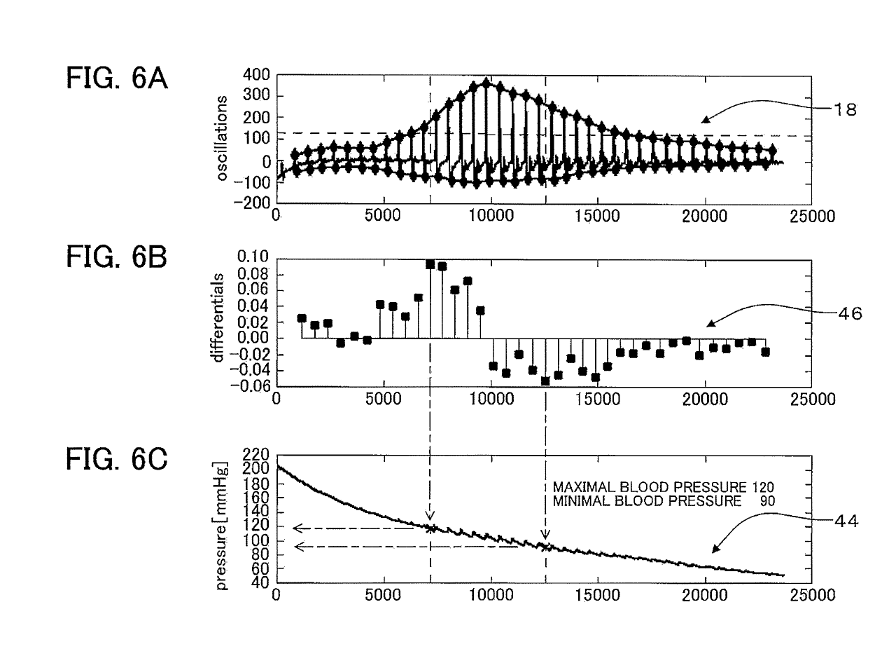

The press mechanism 10 applies an external pressure that causes a radial artery (artery) 16 to produce an oscillometric waveform 18 (see FIG. 6A). The press mechanism 10 applies pressure to a blood vessel by pressing a living body, and can gradually decrease the pressure.

The pressure sensor 12 measures a change in volume with each heartbeat as a change in pressure, converts the change in volume into an electrical signal, and transmits the electrical signal to the control-display section 14.

The control-display section 14 executes a blood pressure calculation algorithm using the resulting oscillometric signal, and displays the results. The control-display section 14 transmits a control signal that controls the pressure applied to the radial artery 16 to the press mechanism 10. The control-display section 14 and the press mechanism 10 are attached to the wrist using a wrist belt 29 that is formed of a flexible plastic or the like, the wrist belt 29 including open ends, and a fastener means (e.g., hook-and-loop fastener (Velcro fastener (registered trademark))) that fastens the ends.

As illustrated in FIG. 2, the artery (radial artery 16) is present in the wrist 20 at a shallow position (i.e., at a depth of 3 to 4 mm from the body surface). A radius 22 is present right under the radial artery 16, so that the pressure applied to the body surface is directly applied to the radial artery 16 without being dispersed. Therefore, the wrist 20 is suitable for blood pressure measurement.

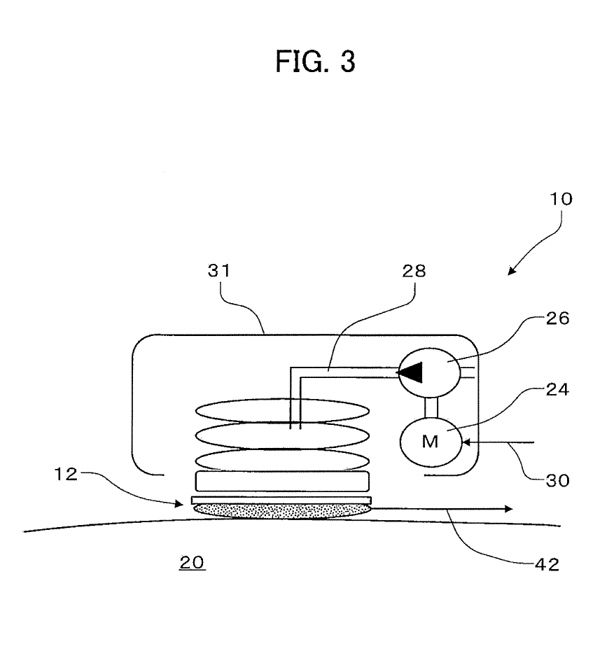

FIG. 3 is a view illustrating the details of the press mechanism 10.

The press mechanism 10 includes a motor 24, a pump 26, an expansion section 28, and a housing 31 that houses each unit.

The motor 24 is controlled based on the control signal transmitted from the control-display section 14 via a control signal line 30. The pump 26 that is driven by the motor 24 supplies air (air from the outside in this example) to the expansion section 28. The expansion section 28 thus presses the pressure sensor 12 against the surface of the wrist 20 from the body surface (side where the radius 22 is present), so that pressure can be applied to the radial artery 16 through a body tissue 34. The expansion section 28 is formed by welding three bag-shaped disks so that the expansion height is 10 mm and the bottom has a radius of 10 mm, for example. The motor 24 has a cylindrical shape having a diameter of 5 mm and a length of 10 mm. The pump 26 has a cylindrical shape having a diameter of 5 mm and a length of 5 mm.

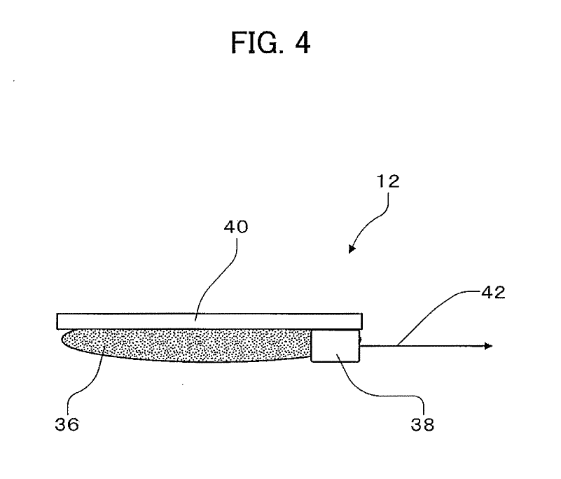

FIG. 4 is a view illustrating the configuration of the pressure sensor 12.

The pressure sensor 12 includes a detection section 36, a pressure-electrical signal converter 38, and a shield 40.

The radial artery 16 changes in volume due to the externally applied pressure and the blood pressure with each heartbeat. The change in volume is detected by the detection section 36 through the body tissue 34. The detection section 36 is filled with an incompressible fluid, and accurately transmits the change in volume detected through the fluid to the pressure-electrical signal converter 38 as a change in pressure.

The pressure-electrical signal converter 38 reads the change in pressure as a change in resistance or the like, converts the change in pressure into an electrical signal, and transmits the electrical signal to the control-display section 14 via a pressure signal line 42.

The amount of fluid in the detection section 36 is managed so that the fluid has dimensions of 15×30×2 mm, for example. The upper side of the detection section 36 (i.e., the side of the detection section 36 opposite to the side that comes in contact with the body tissue 34) is secured on the shield 40 so that a change in pressure can be utilized to a maximum extent. The pressure-electrical signal converter 38 can detect a pressure range including a normal human blood pressure range. For example, the pressure-electrical signal converter 38 may have a detection range of 50 KPa or less.

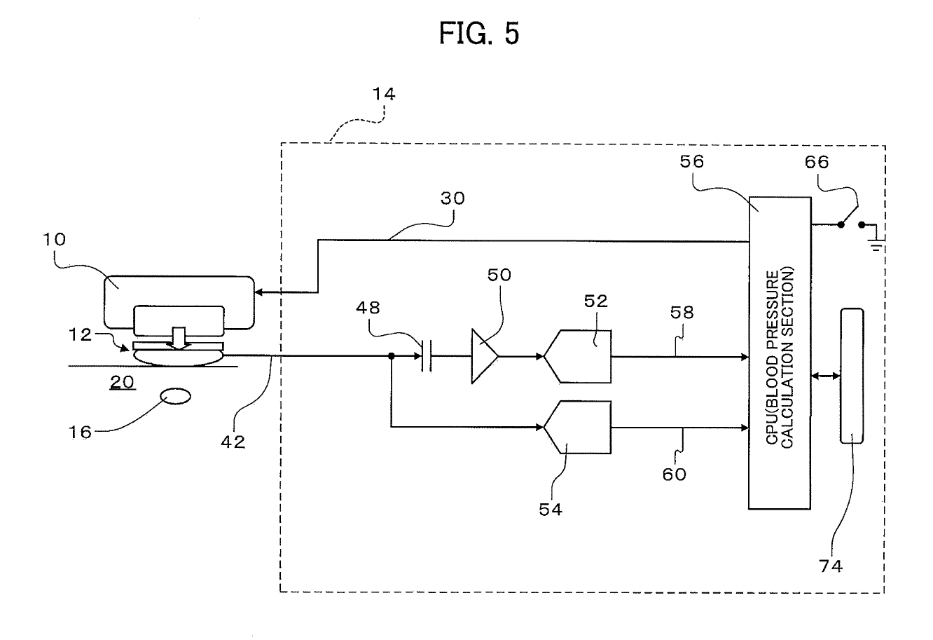

FIG. 5 is a view illustrating the details of the control-display section 14.

The control-display section 14 includes a capacitor 48, an amplifier 50, A/D converters 52 and 54, and a CPU 56 (a blood pressure calculation section).

The pressure signal output from the pressure sensor 12 is input to the control-display section 14 via the pressure signal line 42. The control-display section 14 utilizes the pressure signal as two pieces of information for two different processes. Specifically, the pressure signal is used as a signal that indicates a change in volume (i.e., oscillometric signal). A DC component (direct-current component) is removed from the pressure signal by the capacitor 48. The resulting signal is amplified by the amplifier 50 by a factor of 100, for example, converted into a digital signal by the A/D converter 52, and input to the CPU 56 via a signal line 58. The pressure signal from the pressure sensor 12 is branched from the pressure signal line 42, converted into a digital signal by the A/D converter 54, and input to the CPU 56 via a pressure signal line 60.

A blood pressure calculation section (CPU 56) determines the pressure when a given waveform pattern appears in the waveform of the pulse waves obtained from the pressure sensor 12 when the artery is gradually opened from an occlusion state to be a systolic blood pressure (maximal blood pressure), and determines the pressure when the waveform has the maximum amplitude to be a mean blood pressure.

The blood pressure calculation section calculates the minimal blood pressure (diastolic blood pressure) from the maximal blood pressure and the mean blood pressure. It is known that the systolic blood pressure, the mean blood pressure, and the diastolic blood pressure satisfy the following relational expression.

Mean blood pressure=diastolic blood pressure+(systolic blood pressure−diastolic blood pressure)/3

Therefore, the diastolic blood pressure is calculated by the following expression.

Diastolic blood pressure=(3×mean blood pressure−systolic blood pressure)/2

The blood pressure calculation section included in the blood pressure detection apparatus 2 is implemented by causing the CPU 56 to execute a given program.

Oscillometric Waveform

FIGS. 6A to 6C are views illustrating a standard blood pressure determination algorithm by the oscillometric method according to one embodiment of the invention. FIG. 6A illustrates an oscillometric waveform 18 (peak points of each waveform) obtained when the CPU 56 has performed a waveform process (e.g., noise removal) on a pulse waveform detected by the pressure sensor 12 when externally applying a pressure waveform 44 (see FIG. 6C). The pressure waveform 44 is also detected by the pressure sensor 12, input to the CPU 56 via the pressure signal line 60, and recorded together with the oscillometric waveform 18. A change in volume with a single heartbeat is about several tens of mmV. Since the signal is amplified by the amplifier 50 by a factor of 100, the signal is detected as a change by 2 to 3 V.

An example in which the maximal blood pressure and the minimal blood pressure are determined from a waveform data string based on a differential method (standard algorithm) is described below. A differential waveform 46 (see FIG. 6B) is obtained by differentiating the oscillometric waveform 18. Specifically, the differential waveform 46 is obtained by a difference method that calculates the difference between each peak value of the oscillometric waveform 18 and the adjacent value. The pressure of the pressure signal waveform 44 corresponding to the positive maximum value of the differential waveform 46 corresponds to the maximal blood pressure, and the pressure of the pressure signal waveform 44 corresponding to the negative maximum value of the differential waveform 46 corresponds to the minimal blood pressure. In this example, the maximal blood pressure is determined to be 120 mmHg, and the minimal blood pressure is determined to be 90 mmHg.

Detection of systolic waveform pattern and blood pressure determination method

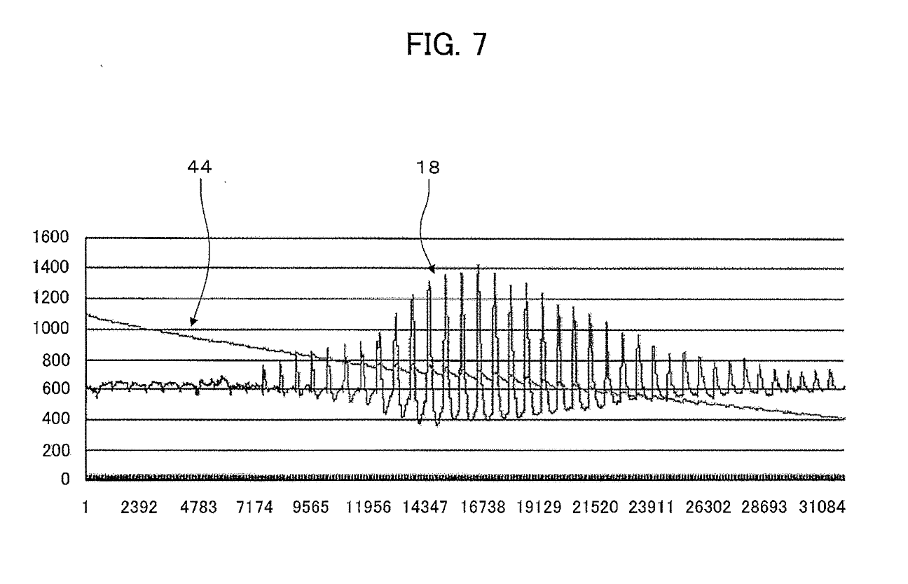

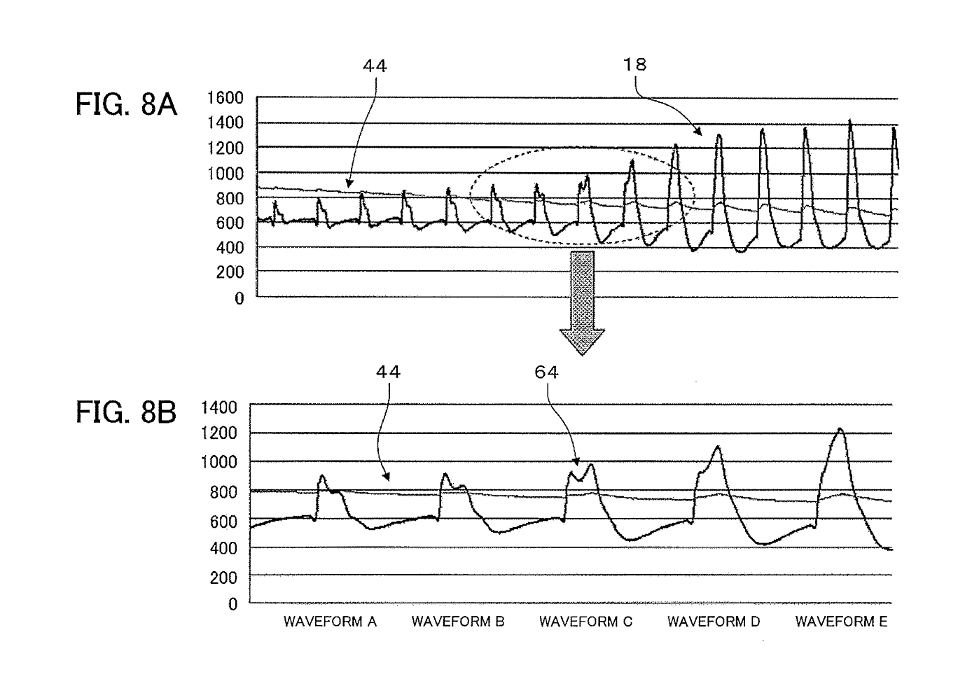

FIGS. 7, 8A, and 8B are views illustrating an oscillometric waveform according to one embodiment of the invention, and a pressure waveform thereof.

The waveforms illustrated in FIG. 7 correspond to the oscillometric waveform 18 and the pressure signal waveform 44 illustrated in FIG. 6. FIG. 8A is an enlarged view of the first half of the waveforms illustrated in FIG. 7 (i.e., an area including the systolic blood pressure), and FIG. 8B is an enlarged view of an area enclosed by a dotted line in FIG. 8A so that a systolic waveform pattern 64 (given waveform pattern) is clearly observed. As is clear from FIG. 8B, when changing the pressure externally applied to the blood vessel, the blood pressure pulse waveform (pulse waves) changes across the systolic waveform pattern 64 (see waveforms A, B, C, D, and E). The waveform C differs from the waveforms A, B, D, and E, and the systolic waveform pattern 64 can be easily identified. In the waveform B, a maximum value among a plurality of maximum values (two maximum values in FIG. 8B) that precedes the other maximum value(s) in time series (i.e., when a higher pressure is applied by the press mechanism 10) is larger than the succeeding maximum value(s) in time series (i.e., when a lower pressure is applied by the press mechanism 10). In the waveform C, a maximum value among a plurality of maximum values that precedes the other maximum value(s) in time series (i.e., when a higher pressure is applied by the press mechanism 10) is smaller than the succeeding maximum value(s) in time series (i.e., when a lower pressure is applied by the press mechanism 10). Specifically, the relationship between the plurality of maximum values of the waveform C differs from (is the reverse of) that of the waveform B. Therefore, the systolic waveform pattern 64 can be determined based on whether or not the relationship between the maximum values of the waveform has changed.

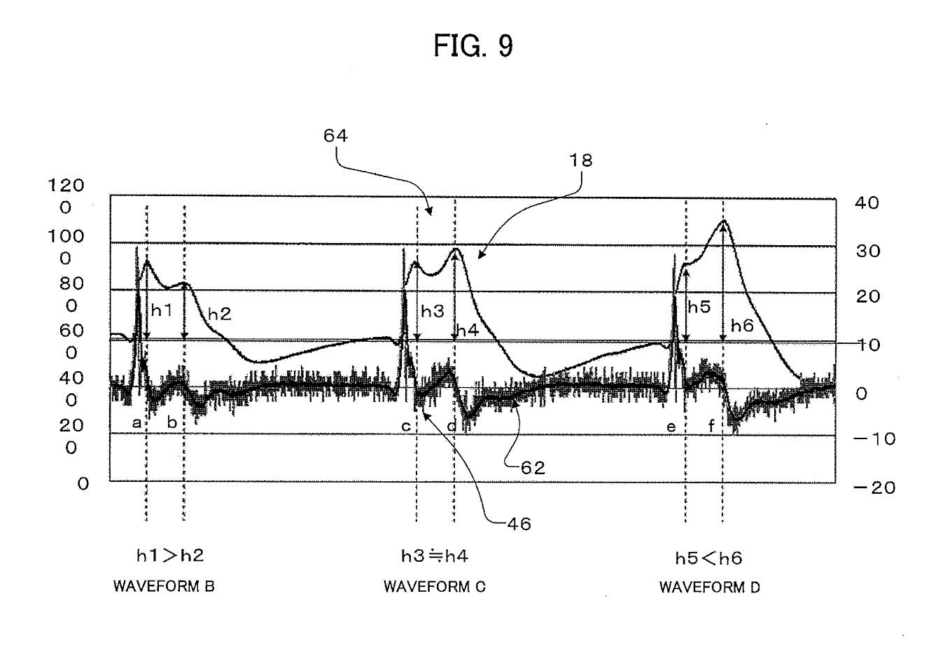

FIG. 9 is a view illustrating an oscillometric waveform according to one embodiment of the invention, and a differential waveform thereof.

The method of extracting the systolic waveform pattern 64 is described in more detail below. The differential waveform 46 illustrated in FIG. 9 is obtained by differentiating the oscillometric waveform 18, and includes a significant systolic waveform pattern 64. The above difference method may be used instead of the differentiation method. As illustrated in FIG. 9, large noise is normally superimposed on the differential waveform 46, and the differential waveform 46 is normally used after removing noise (smoothing the waveform). For example, the sum of three successive signal values is calculated, and divided by the number of pieces of data (moving average method). In this case, the phase must be restored after the process.

a, b, c, and d indicate points where the value of the oscillometric waveform 18 becomes a maximum among points where the slope of a smoothed waveform 62 obtained by smoothing the differential waveform 46 is 0. Regarding the difference from the average value of the oscillometric waveform 18, the waveform B satisfies the relationship “h1>h2”, and the waveform C satisfies the relationship “h3<h4” in the example illustrated in FIG. 9. Specifically, the relationship between the plurality of maximum values of the waveform C differs from (is the reverse of) that of the waveform B. Therefore, it can be easily determined that the waveform C includes the systolic waveform pattern 64.

The systolic waveform pattern 64 can be normally detected by the above method. However, a clear systolic waveform pattern 64 may not be obtained from the temporal relationship between the decompression speed and the blood pressure pulse waveform. This applies to a case where the waveform D is directly generated from the waveform B without generating the waveform C (see FIG. 9), for example. In this case, the pressure (applied pressure) detected by the pressure sensor 12 which corresponds to the waveform B, and the pressure (applied pressure) detected by the pressure sensor 12 and corresponds to the waveform D are determined, and the median value therebetween is determined to be the systolic blood pressure. Therefore, the blood pressure can be determined without measuring the entire oscillometric waveform 18 (i.e., the advantage of the invention is not impaired).

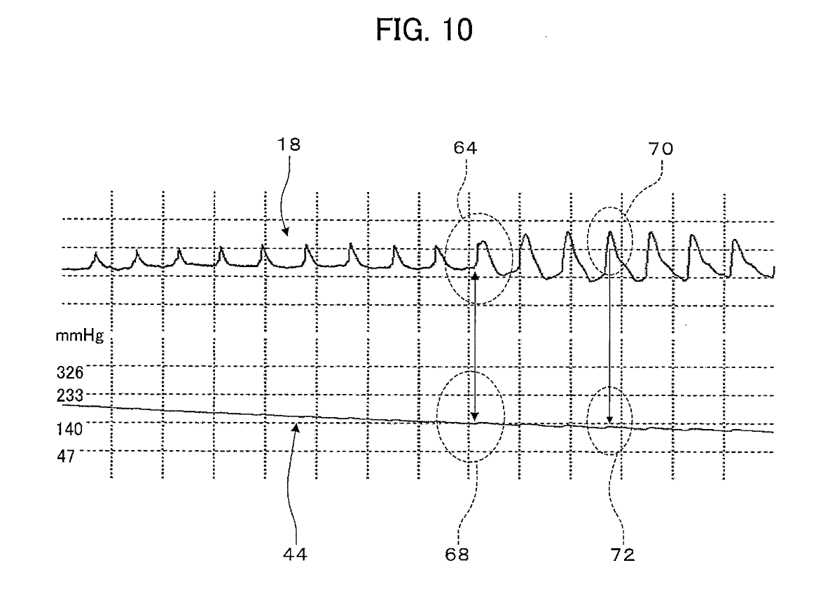

FIG. 10 is a view illustrating the systolic waveform pattern 64 included in the oscillometric waveform 18 according to one embodiment of the invention. When collecting and analyzing many oscillometric waveforms 18 by small blood pressure measurement technology, it was found that the oscillometric waveform 18 has a specific waveform (systolic waveform pattern 64) when the pressure (applied pressure) detected by the pressure sensor 12 is (almost) equal to the systolic blood pressure 68 of the pressure signal waveform 44. The pressure (applied pressure) (i.e., systolic blood pressure 68) detected by the pressure sensor 12 which corresponds to the systolic waveform pattern 64 indicates the maximal blood pressure. Note that the applied pressure corresponding to the maximum value of the systolic waveform pattern 64 that precedes or succeeds in time series may be determined to be the maximal blood pressure. The average value of the applied pressure corresponding to the maximum value that precedes in time series and the applied pressure corresponding to the maximum value that succeeds in time series may be determined to be the maximal blood pressure.

The waveform illustrated in FIG. 10 is not observed by a cuff method, but is observed when applying pressure to the radial artery 16 by locally pressing a skin area (body surface) positioned above the radial artery 16, and measuring the change in volume of the blood vessel with each heartbeat using the small pressure sensor 12. The pressure (applied pressure) (i.e., mean blood pressure 72) detected by the pressure sensor 12 when the oscillometric waveform 18 has the maximum amplitude (mean blood pressure waveform 70) indicates the mean blood pressure. Specifically, the applied pressure when an oscillometric waveform has the maximum amplitude is medically defined as the mean blood pressure.

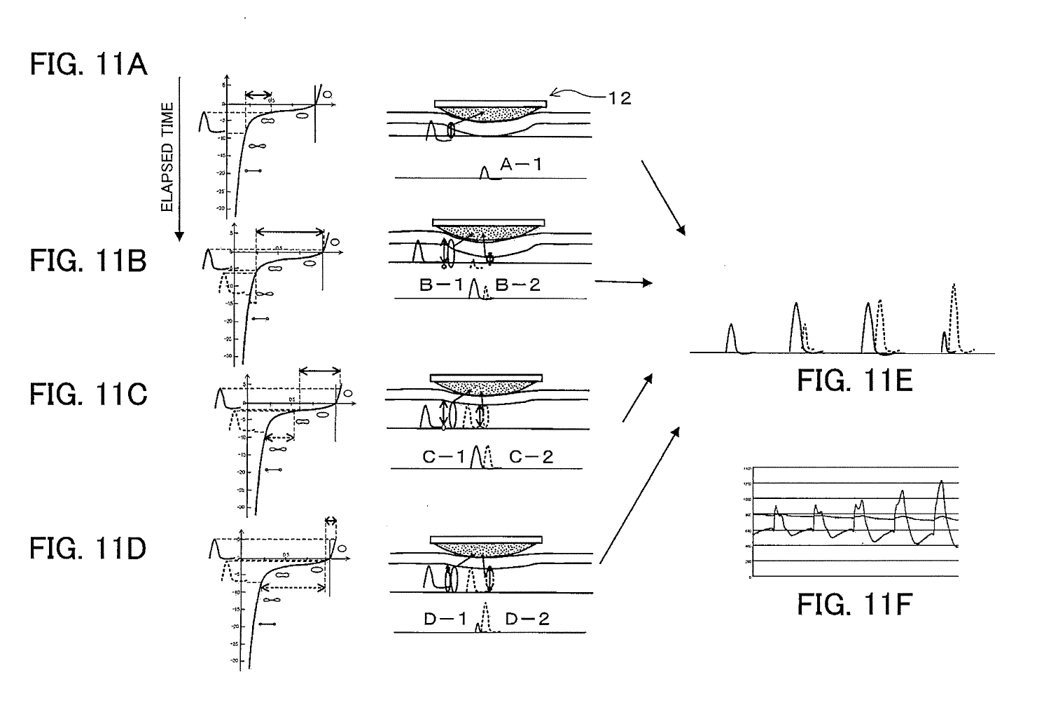

FIGS. 11A to 11F are views illustrating the systolic waveform pattern 64 included in the oscillometric waveform 18 according to one embodiment of the invention. The left side of FIGS. 11A to 11D indicates the relationship between the internal-external pressure difference based on the tube law and the cross-sectional area of the blood vessel in time series. Since a change in cross-sectional area differs depending on the internal-external pressure difference, the pressure detected by the pressure sensor 12 differs depending on the change range even if the change in pressure (pulse) is the same. Since the pressure applied to the blood vessel differs between the peripheral area and the center area of the pressure sensor 12, the difference in change in pressure appears as a temporal difference. The size of the waveform becomes equal due to a change, and is reversed. The detected pressure differs between the peripheral area and the center area of the pressure sensor 12 even if the pressure is the same. Moreover, the peripheral area of the pressure sensor 12 has low sensitivity, and the center area of the pressure sensor 12 has high sensitivity.

A change that occurs in time series is described below. As illustrated in FIG. 11A, since the blood vessel that comes in contact with the center area of the pressure sensor 12 is occluded when the applied pressure is high, a signal is not generated from the center area of the pressure sensor 12, and a signal is generated from the peripheral area of the pressure sensor as a small waveform A-1 according to the tube law.

As illustrated in FIG. 11B, the blood vessel that comes in contact with the center area of the pressure sensor 12 is opened to some extent when the applied pressure is reduced, and a signal is generated from the center area of the pressure sensor 12 as a small waveform B-2 according to the tube law. A signal is generated from the peripheral area of the pressure sensor as a medium waveform B-1 according to the tube law. The medium waveform B-1 precedes the small waveform B-2. This is because vibrations applied to the peripheral area of the pressure sensor 12 start earlier than those applied to the center area.

As illustrated in FIG. 11C, the blood vessel that comes in contact with the center area of the pressure sensor 12 is further opened when the applied pressure is further reduced, and a signal is generated from the center area of the pressure sensor 12 as a medium waveform C-2 according to the tube law. A signal is generated from the peripheral area of the pressure sensor as a medium waveform C-1 according to the tube law. Specifically, the waveforms of the center area and the peripheral area of the pressure sensor 12 become almost equal.

As illustrated in FIG. 11D, the blood vessel that comes in contact with the center area of the pressure sensor 12 is further opened when the applied pressure is further reduced, and a signal is generated from the center area of the pressure sensor 12 as a large waveform D-2 according to the tube law. A signal is generated from the peripheral area of the pressure sensor as a small waveform D-1 according to the tube law.

FIG. 11E illustrates these waveforms in time series, and FIG. 11F illustrates the actual waveform.

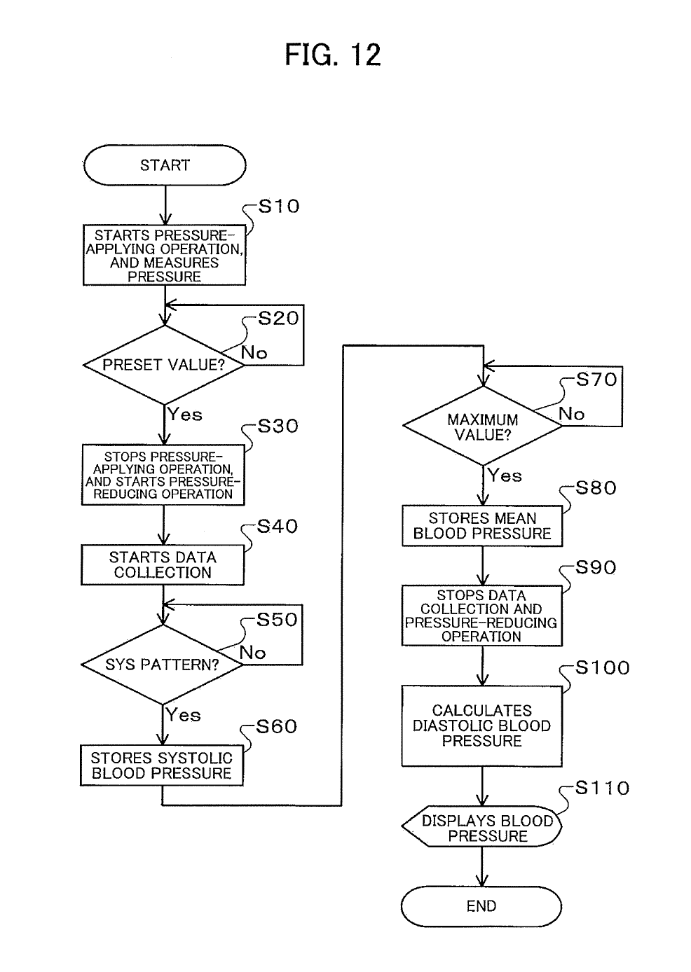

FIG. 12 is a flowchart illustrating the entire operation (process) according to one embodiment of the invention. The entire operation (process) is described below with reference to FIG. 12.

The blood pressure detection apparatus 2 starts to operate when a switch 66 of the control-display section 14 has been pressed (step S10). When the CPU 56 has detected that the switch 66 has been pressed, the CPU 56 instructs the press mechanism 10 to start a pressure-applying operation via the control signal line 30. The press mechanism 10 then activates the motor 24 and the pump 26 so that air is supplied to the expansion section 28. The CPU 56 starts measurement of the pressure detected by the pressure sensor 12 and input via the pressure signal lines 42 and 60.

The CPU 56 then determines whether or not the pressure detected by the pressure sensor 12 is equal to or greater than a preset value (e.g., 200 mmHg) (step S20). When the CPU 56 has determined that the pressure detected by the pressure sensor 12 is less than 200 mmHg (No), the CPU 56 continues to determine whether or not the pressure detected by the pressure sensor 12 is equal to or greater than 200 mmHg. When the CPU 56 has determined that the pressure detected by the pressure sensor 12 is equal to or greater than 200 mmHg (Yes), the CPU 56 then performs a step S30.

Specifically, the CPU 56 instructs the press mechanism 10 to stop the pressure-applying operation and start a pressure-reducing operation via the control signal line 30 (step S30). The pump 26 included in the press mechanism then stops the pressure-applying operation, and starts the pressure-reducing operation. The pressure-reducing operation is performed at a constant rate of 3 mmHg per second.

The CPU 56 then starts measurement (700 times per second) of the oscillometric signal input from the signal line 58 (step S40). The oscillometric waveform 18 is obtained by arranging the measured values in time series. The CPU 56 stores a signal received from the pressure sensor 12 in a memory (not shown) while sequentially receiving a signal from the pressure sensor 12 to generate the oscillometric waveform 18.

The CPU 56 then determines the shape of the oscillometric waveform 18 (step S50). When the determined shape of the oscillometric waveform 18 is not the systolic waveform pattern 64 (see FIG. 10) (No), the CPU 56 determines the shape of the next oscillometric waveform 18. When the determined shape of the oscillometric waveform 18 is the systolic waveform pattern 64 (Yes), the CPU 56 then performs a step S60.

The CPU 56 then stores the pressure (applied pressure) (i.e., systolic blood pressure 68) detected by the pressure sensor 12 which corresponds to the systolic waveform pattern 64 (step S60).

The CPU 56 then determines whether or not the oscillometric waveform 18 has the maximum amplitude (step S70). When the CPU 56 has determined that the oscillometric waveform 18 does not have the maximum amplitude (No), the CPU 56 determines whether or not the next oscillometric waveform 18 has the maximum amplitude. When the CPU 56 has determined that the oscillometric waveform 18 has the maximum amplitude (Yes), the CPU 56 then performs a step S80.

The CPU 56 then stores the pressure (applied pressure) (i.e., mean blood pressure 72) detected by the pressure sensor 12 when the oscillometric waveform 18 has the maximum amplitude (mean blood pressure waveform 70) in the memory (step S80). The CPU 56 has thus determined the systolic blood pressure 68 and the mean blood pressure 72.

The CPU 56 then stops the measurement of the oscillometric signal input via the signal line 58 (step S90). The CPU 56 instructs the press mechanism 10 to stop the pressure-reducing operation via the control signal line 30. The pump 26 included in the press mechanism then stops the pressure-reducing operation.

The CPU 56 then calculates the diastolic blood pressure (minimal blood pressure) from the systolic blood pressure 68 and the mean blood pressure 72 using the blood pressure calculation section (step S100).

The CPU 56 then displays the systolic blood pressure 68 and the diastolic blood pressure on a display device 74, and finishes the process (step S110).

According to the above embodiments, an accurate blood pressure can be quickly determined. When the blood pressure detection apparatus is used as a wearable sphygmomanometer that allows arbitrary blood pressure measurement, the blood pressure can be frequently measured without causing inconvenience to the user, so that the blood pressure can be monitored advantageously.

Modifications

An example in which the applied pressure when the systolic waveform pattern 64 occurs is determined to be the maximal blood pressure while reducing the applied pressure has been described above. Note that the applied pressure when the systolic waveform pattern 64 occurs may be determined to be the maximal blood pressure while increasing the applied pressure. Specifically, the blood pressure calculation section may determine the pressure when a given waveform pattern appears in the waveform of the pulse waves obtained from the pressure sensor 12 when the artery is gradually occluded from an open state to be the systolic blood pressure (maximal blood pressure), and may determine the pressure when the waveform has the maximum amplitude to be the mean blood pressure.

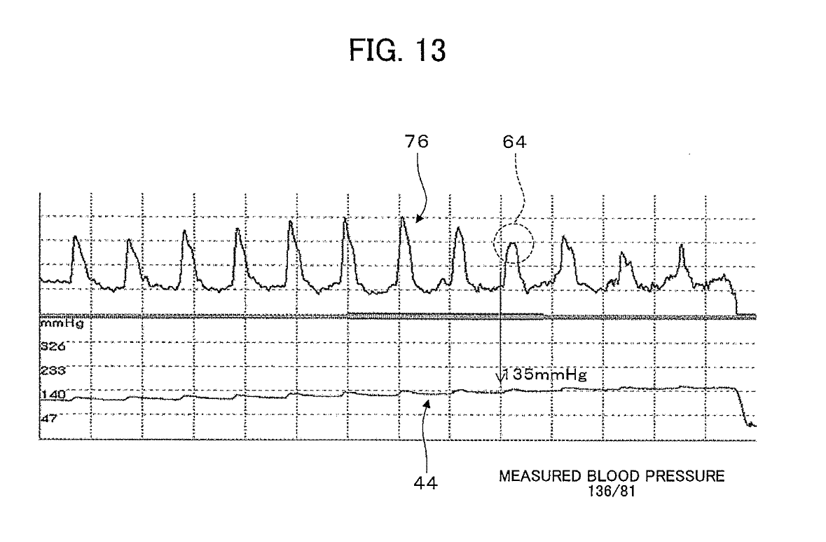

FIG. 13 is a view illustrating a systolic waveform pattern included in an oscillometric waveform according to this modification. FIG. 13 illustrates a pressure signal waveform 44 of pressure applied to the blood vessel (lower side), and a pulse waveform 76 detected when applying the pressure to the blood vessel (upper side). A systolic waveform pattern 64 included in the pulse waveform 76 differs from other waveforms. In the systolic waveform pattern 64, a maximum value among a plurality of maximum values that precedes the other maximum value(s) in time series (i.e., when a lower pressure is applied by the press mechanism 10) is larger than the succeeding maximum value(s) in time series (i.e., when a higher pressure is applied by the press mechanism 10) (the details thereof are not illustrated in FIG. 13). In the pulse waveform that precedes the systolic waveform pattern 64, a maximum value among a plurality of maximum values that precedes the other maximum value(s) in time series (i.e., when a lower pressure is applied by the press mechanism 10) is smaller than the succeeding maximum value(s) in time series (i.e., when a higher pressure is applied by the press mechanism 10). Specifically, the relationship between the maximum values of the systolic waveform pattern 64 differs from (is the reverse of) the relationship between the maximum values of the pulse waveform that precedes the systolic waveform pattern 64. Therefore, the systolic waveform pattern 64 can be determined based on whether or not the relationship between the maximum values of the waveform has changed. The pressure of the pressure signal waveform 44 corresponding to the systolic waveform pattern 64 is 135 mmHg, which is very close to the maximal blood pressure (136 mmHg) measured by another sphygmomanometer. Therefore, the maximal blood pressure can be conveniently determined without increasing and decreasing the applied pressure as in the case of using a normal sphygmomanometer.

An example in which the blood pressure calculation section determines the pressure when the waveform of the pulse waves obtained from the pressure sensor 12 when the artery is gradually opened from an occlusion state has the maximum amplitude to be the mean blood pressure has been described above. Note that the blood pressure calculation means may determine the pressure when the waveform has the maximum value to be the mean blood pressure.

Although only some embodiments of the invention have been described in detail above, those skilled in the art would readily appreciate that many modifications are possible in the embodiments without materially departing from the novel teachings and advantages of the invention. Accordingly, such modifications are intended to be included within the scope of the invention.

What is claimed is:

1. A blood pressure detection apparatus comprising:
a press mechanism that applies pressure to a blood vessel by pressing a living body, and can gradually decrease the pressure;
a pressure sensor that detects a change in pressure of the blood vessel caused by a change in the pressure applied by the press mechanism; and
a blood pressure calculation section that determines the pressure applied by the press mechanism when a given waveform pattern appears in a waveform that indicates a change in pressure of the blood vessel to be a maximal blood pressure, determines the pressure applied by the press mechanism when the waveform has a maximum amplitude to be a mean blood pressure, and calculates a minimal blood pressure using the maximal blood pressure and the mean blood pressure.

2. The blood pressure detection apparatus as defined in claim 1,
the given waveform pattern being a waveform that indicates pulse waves that include a first maximum value, and a second maximum value that is obtained when the pressure applied by the press mechanism is lower than that when the first maximum value is obtained, the second maximum value being larger than the first maximum value.

3. The blood pressure detection apparatus as defined in claim 1,
the press mechanism gradually opening an artery from an occlusion state.

4. The blood pressure detection apparatus as defined in claim 1,
the press mechanism gradually occluding an artery from an open state.

5. The blood pressure detection apparatus as defined in claim 3,
the artery being a radial artery.

6. A blood pressure detection method comprising:
applying pressure to a blood vessel by pressing a living body;
gradually decreasing the pressure applied to the blood vessel;
detecting a change in pressure of the blood vessel caused by a change in the pressure applied to the blood vessel; and
determining the pressure applied to the blood vessel when a given waveform pattern appears in a waveform that indicates a change in pressure of the blood vessel to be a maximal blood pressure, determining the pressure applied to the blood vessel when the waveform has a maximum amplitude to be a mean blood pressure, and calculating a minimal blood pressure using the maximal blood pressure and the mean blood pressure.
